(19) United States
(12) Patent Application Publication
(10) Pub. No.: US 2023/0139806 A1
(43) Pub. Date: May 4, 2023

(54) SELENIDE DERIVATIVES OF FRAGRANCE COMPOUNDS

(71) Applicant: SYMRISE AG, Holzminden (DE)

(72) Inventors: Antonio RUIZ SANCHEZ, Granada (ES); Ricardo RODRIGUEZ FERROL, Granada (ES)

(21) Appl. No.: 17/795,731

(22) PCT Filed: Jan. 28, 2021
(86) PCT No.: PCT/EP2021/052006

§ 371 Date: Jul. 27, 2022

(30) Foreign Application Priority Data
Jan. 28, 2020 (EP) 20382050.1

(51) Int. Cl. A61K 8/23; A61K 8/35; A61K 8/36; A61Q 13/00

(52) U.S. Cl. CPC A61K 8/23; A61K 8/35; A61K 8/36; A61Q 13/00

(57) ABSTRACT

There is provided the use of selenide derivatives of fragrance compounds comprising an α,β-unsaturated aldehyde moiety, an α,β-unsaturated ketone moiety, an α,β-unsaturated ester moiety, an α,β-unsaturated lactone moiety or an α,β-unsaturated carboxylic acid moiety in consumer products. There are further provided methods for the synthesis of selenide derivatives, certain selenide derivatives, consumer products comprising the selenide derivatives and methods of perfuming an article or surface using such derivatives.

The present disclosure relates to the use of selenide (selenoether) derivatives of fragrance compounds comprising an a,β-unsaturated aldehyde, ketone, ester, lactone or carboxylic acid moiety for the controlled release of the fragrance compounds.

It also relates to methods for synthesis of selenide derivatives of fragrance compounds comprising an a,β-unsaturated aldehyde, ketone, ester, lactone or carboxylic acid moiety as well as the selenide derivatives themselves. The invention further relates to consumer products comprising the selenide derivatives and methods of perfuming an article or surface using such derivatives.

The perfume industry has long held a need for compounds or formulations which can sustain or facilitate the longevity of fragrance of fragrance compounds used in consumer products such as a fine perfumes, fabric softeners, hair care products, skin care products and the like.

The need is particularly felt in the washing of fabrics or textiles because many of the fragrance compounds suitable for this application are known to lack substantivity or tenacity on laundry or do not remain on laundry when rinsed.

One approach to addressing this need is based upon the use of derivatives of fragrance compounds comprising an a,β-unsaturated aldehyde, ketone, ester, lactone or carboxylic acid moiety and subsequent elimination of chemical species from the derivative in ambient environment to regenerate the starting fragrance compounds. Typical of this approach are the disclosures of International Patent Applications WO 03/049666 A2 and WO 2015/032885 A2. In these disclosures, the preparation of β-sulfide derivatives (amongst others) of fragrance compounds comprising an a,β-unsaturated aldehyde, ketone, or ester moiety and their use for the controlled release of the fragrance compound on such surfaces as textile surfaces, hard surfaces, hair or skin is disclosed.

One problem with the use of β-sulfide derivatives for the controlled release of these fragrance compounds is that the derivative is normally contaminated with volatile thiol.

The volatile thiol is foul smelling and extremely difficult to remove from the β-sulfide derivative when purified by standard methods. It persists during the controlled release of the fragrance compound and obscures or detracts from the fragrance note of the released fragrance compound.

It has now surprisingly been found that selenide derivatives of fragrance compounds comprising an a,β-unsaturated aldehyde, ketone, ester, lactone or carboxylic acid moiety may provide for a longer-lasting controlled release of the fragrance compound as compared to the corresponding sulphur derivatives.

It has also been found that the selenide derivatives may provide for a cleaner (or clearer) fragrance note of the released fragrance compound as compared to the use of β-sulfide derivative.

Further, the selenide derivatives themselves have a fragrance note substantially similar to that of the fragrance compounds - even when obtained in crude form.

Accordingly, in a first aspect, the present disclosure provides for the of a selenide derivative of a fragrance compound comprising an a,β-unsaturated aldehyde moiety, an a,β-unsaturated ketone moiety, an a,β-unsaturated ester moiety, an a,β-unsaturated lactone moiety or an a,β-unsaturated carboxylic acid moiety in a consumer product.

In a second aspect, the present disclosure provides a consumer product comprising a selenide derivative of a fragrance compound comprising an a,β-unsaturated aldehyde moiety, an a,β-unsaturated ketone moiety, an a,β-unsaturated ester moiety, an a,β-unsaturated lactone moiety or an a,β-unsaturated carboxylic acid moiety.

In a third aspect, the present disclosure provides a method of perfuming an article or surface comprising treating the article or surface with a selenide derivative of a fragrance compound comprising an a,β-unsaturated aldehyde moiety, an a,β-unsaturated ketone moiety, an a,β-unsaturated ester moiety, an a,β-unsaturated lactone moiety, or an a,β-unsaturated carboxylic acid moiety. Suitable articles or surfaces comprise textiles, hard surfaces, hair and skin.

References herein to a fragrance compound include references to a chemical compound which is used in the perfumery industry. The chemical compound may, in particular, be recognised as imparting a hedonic effect or as modifying in positive or pleasant way the odour of a composition. Alternatively, it may be a preservative, for example, an isothiazolone (such as is described in WO 2015/032885 A2), which does not impart such an effect.

References herein to a fragrance compound comprising an a,β-unsaturated aldehyde, ketone, ester, lactone or carboxylic acid moiety are references to a fragrance compound containing an aldehyde, ketone, ester, lactone or carboxylic acid group which is conjugated with a carbon-carbon double bond neighbouring to the carbonyl moiety of the aldehyde, ketone, ester, lactone or carboxylic acid group.

Many such fragrance compounds can be found in the appropriate reference sources for perfumery including, but not limited to, “Perfume and Flavor Chemicals” by Arctander, S. 1969, Montclair, New Jersey, US (and its more recent versions).

Note that the derivatisation of a,β-unsaturated aldehydes, ketones, esters, lactones or carboxylic acids by selenide may, for example, be achieved by Michael addition of an alkyl or other selenide ion. The addition results in the incorporation of an alkyl selenide (or other selenide) group at the carbon atom of the carbon-carbon double bond furthest (β to) from the carbonyl moiety of the aldehyde, ketone, ester, lactone or carboxylic acid group.

Note also that the derivatisation of a,β-unsaturated aldehydes, ketones, esters, lactones or carboxylic acids by selenide may be achieved by addition of hydrogen free radical and quenching of the addition product with an alkyl or other selenol. The reaction results in the incorporation of an alkyl selenide (or other selenide) group at the carbon atom of the carbon-carbon double bond nearest (a to) the carbonyl moiety of the aldehyde, ketone, ester, lactone or carboxylic acid group.

Note further, that whilst the a,β-unsaturated aldehyde, ketone, ester, lactone or carboxylic acid may contain one or more further carbon-carbon double bonds, preferably none of the other double bonds are conjugated with the carbon-carbon double bond conjugated with the aldehyde, ketone, ester, lactone or carboxylic acid group.

References herein to a-selenide derivatives and β-selenide derivatives of a fragrance compound comprising a,β-unsaturated aldehyde, ketone, ester, lactone or carboxylic acid moiety will be understood accordingly.

Note also that the selenide derivatives may be optically active and, in particular, may comprise a racemate, a single enantiomer, a mixture of diastereoisomers or a single diastereoisomer.

In any case, the selenide derivative should be susceptible to regenerating the fragrance compound on exposure to ambient environment.

The elimination may, for example, occur by oxidation of the selenide group followed by an elimination of the oxidation product (for example, a selenoxide or a selenone).

Note that, although the rate of oxidation of the alkyl or other selenide in ambient environment depends in large part on the susceptibility of selenium to oxidation, it may also be dependent on the size and nature of the alkyl or other group.

Furthermore, the rate of elimination of the oxidation product of the selenide is to a large extent determined by the relative stability of the a,β-unsaturated aldehyde, ketone or ester as compared to the oxidation product.

Accordingly, it will be appreciated that the selenide derivative not only provides for a sustained release of the fragrance compound - but also for a controlled release which, to a certain extent, can be determined by a selection of the alkyl (or other) group for the selenide derivative.

Accordingly, the use of the specified selenide derivatives in consumer products and for perfuming articles and surfaces provides for the release (regeneration) of the fragrance compound for a period of at least 3 days, and generally much longer, such as for up to 5 weeks or longer. In some cases, the period of fragrance release may be at least 5 days, at least 10 days, at least 21 days or at least 35 days.

Furthermore, the selenide derivatives of this disclosure begin to release the base fragrance compound as soon as they come into contact with oxygen, and therefore when they are used in consumer products or for perfuming articles or surfaces they will effectively provide immediate release of the fragrance compound on application of the consumer product to an article or surface.

In general, once fragrance release begins it continues at a relatively steady state for a significant proportion of the total release period.

In certain embodiments of the uses, consumer products and methods of the present disclosure, the selenide derivative has chemical formula:

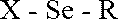

wherein X is a group generating a fragrance compound comprising an a,β-unsaturated aldehyde, ketone, ester, lactone or carboxylic acid moiety by elimination of an ambient oxidation product of the —Se—R group; and wherein R is a C₁₋₂₀ linear, cyclic or branched chain alkyl group or a C₆₋₁₀ unsubstituted or substituted aryl or heteroaryl group, wherein the substituent is a C₁₋₁₂ linear or branched chain alkyl group, for example, a C₁₋₃ alkyl group; preferably R is a C₅₋₁₆ linear alkyl group, more preferably a C₁₀₋₁₄ linear alkyl group, most preferably a C₁₂ linear alkyl group.

In certain embodiments of the uses, consumer products and methods of the present disclosure, the selenide derivative has chemical formula:

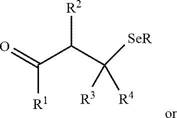

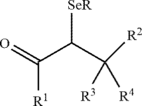

wherein R¹ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group or a C₁₋₁₂ alkoxy group; or
R¹ is a group — OR⁵, wherein R⁵ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group, or a C₁₋₁₂ alkoxy group; and
R², R³, R⁴ are independently a hydrogen atom; a C₁₋₂₀ linear or branched chain alkyl, alkenyl alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group optionally substituted with one or more of a substituent comprising a C₁₋₁₂ linear or branched chain alkyl group, a hydroxy or a C₁₋₁₂ alkoxy group; or
at least two of R¹, R², R³ and R⁴ are bonded together to form a saturated or unsaturated C₅₋₂₀ carbocyclic ring including the carbon atoms to which the at least two of R¹, R², R³, and R⁴ are attached, the ring being optionally substituted with one or more of a substituent comprising a C₁₋₁₂ linear, cyclic or branched chain alkyl or alkene group; and the remainder of R¹, R², R³ and R⁴ are as described above; or
when R¹ is the group —OR⁵, R⁵ and one of R², R³ and R⁴ are bonded together to form a saturated or unsaturated C₄₋₁₉ lactone ring including the carbon atoms to which the at least two of R², R³, R⁴and R⁵ are attached, the ring being optionally substituted with one or more of a substituent comprising a C₁₋₁₂ linear, cyclic or branched chain alkyl or alkene group; and the remainder of R², R³, R⁴ and R⁵ are as described above.

In certain embodiments of the uses, consumer products and methods of the present disclosure, R¹ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group or a C₁₋₁₂ alkoxy group; and R², R³, R⁴ are independently a hydrogen atom; a C₁₋₂₀ linear or branched chain alkyl, alkenyl alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group optionally substituted with one or more of a substituent comprising a C₁₋₁₂ linear or branched chain alkyl group, a hydroxy or a C₁₋₁₂ alkoxy group.

In other embodiments, R¹ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group or a C₁₋₁₂ alkoxy group; and at least two of R¹, R², R³ and R⁴ are bonded together to form a saturated or unsaturated C₅₋₂₀ carbocyclic ring including the carbon atoms to which the at least two of R¹, R², R³, and R⁴ are attached, the ring being optionally substituted with one or more of a substituent comprising a C₁₋₁₂ linear, cyclic or branched chain alkyl or alkene group; and the remainder of R¹, R², R³ and R⁴ are as described above.

In still further embodiments, R¹ is a group — OR⁵, wherein R⁵ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group or a hydroxy group, or a C₁₋₁₂ alkoxy group; and R⁵ and one of R², R³ and R⁴ are bonded together to form a saturated or unsaturated C₄₋₁₉ lactone ring including the carbon atoms to which the at least two of R², R³, R⁴ and R⁵ are attached, the ring being optionally substituted with one or more of a substituent comprising a C₁₋₁₂ linear, cyclic or branched chain alkyl or alkene group; and the remainder of R², R³ and R⁴ are as described above.

Note here that the remainder of R², R³ and R⁴ may be bonded together so as to form an unsaturated carbocyclic ring as described above.

R¹ may, for example, comprise a C₁₋₁₅ or C₁₋₁₂ or C₁₋₆ linear or branched chain alkyl, alkenyl, alkadienyl or alkynyl group. It may, in particular, comprise methyl, ethyl, n-propyl, allyl, or ethynyl. Alternatively, R¹ may comprise a C₇-₁₂ linear or branched chain group. When R¹ is a C₅₋₁₀ cyclic alkyl or cyclic alkene it may, in particular, comprise a fully saturated C₅ or C₆ cyclic ring or a C₅ or C₆ cyclic ring having one or more carbon-carbon double bonds. The substituent for these groups may, in particular, comprise a C₁₋₆ or a C₁₋₄ linear or branched chain alkyl group. The substituent may in particular be chosen amongst the group consisting of methyl, ethyl, n-propyl, i-propyl, s-butyl, t-butyl or n-butyl groups.

When R¹ is a C₆₋₁₀ aryl group, it may, for example, comprise a phenyl group or a naphthalenyl group. The substituent for these groups may, in particular, comprise a C₁₋₆ or C₁₋₄ linear or branched chain alkyl group such as those described above. Alternatively, the substituent may be a C₁₋₆ or C₁₋₄ alkoxy group and, in particular, a methoxy or ethoxy group.

When at least two of R¹, R², R³ and R⁴ are bonded together to form a saturated or unsaturated C₅₋₂₀ carbocyclic ring including the carbon atoms to which the at least two of R¹, R², R³, and R⁴ are attached, the ring may be a five, six, seven or eight membered ring.

The carbocyclic ring may, for example, include one or more carbon-carbon double bonds. The at least two of R¹, R², R³ and R⁴ may bond together so as to form more than one carbocyclic ring, for example, two or three carbocyclic rings, which may, in particular, be five or six membered rings. When more than one carbocyclic ring is formed one or more of these carbocyclic rings may include one or more carbon-carbon double bonds.

The at least two of R¹, R², R³ and R⁴ may bond together so as to form two or more fused carbocyclic rings, for example a C₁₀ fused carbocyclic ring comprising two C₆ rings. Such fused carbocyclic rings may be saturated or may include one or more carbon-carbon double bonds.

The substituent for these groups may, in particular, comprise a C₁₋₈, C₁₋₆ or C₁₋₄ linear or branched chain alkyl group such as described above. Alternatively, the substituent may comprise a C₁₋₆ or C₁₋₄ alkene group, in particular, an —CH(CH₃)CH₂ group. The substituent may also comprise a C₁₋₆ cyclic alkyl group, for example, C₁₋₄ cyclic alkyl group.

In some embodiments, R² and R³ may be bonded together (with R⁴ being, for example, hydrogen atom). The five- or six-membered carbocyclic ring may comprise one or more alkylene groups (—CH₂—), for example, two, three or four -which may be substituted by one or more of a C₁₋₅ alkyl group, such as methyl.

The alkylene groups may be substituted by one or more further unsubstituted or C₁₋₅ alkyl substituted alkylene groups forming one or more of an additional five-or six-membered carbocyclic ring. In that case, the a or β-selenide derivative comprises two or three fused five- or six-membered carbocyclic rings.

In other embodiments, R¹ and R³ may be bonded together (with R⁴ being, for example, hydrogen atom). The five or six membered carbocyclic ring may comprise one or more alkylene groups (—CH₂—), for example, two, three or four -which may be substituted by one or more of a C₁₋₄ alkyl group, for example, methyl.

The alkylene groups may be substituted by one or more further unsubstituted or C₁₋₅ alkyl or —CH₂— alkylene substituted alkylene groups forming an additional five-or six-membered carbocyclic ring. In that case, the a- or β-selenide derivative may comprise a spiro compound of two carbocyclic rings.

When R¹ is the group —OR⁵ the at least two of R², R³, R⁴ and R⁵ may bond together to form a saturated or unsaturated C₄₋₁₉ lactone ring including the carbon atoms to which the at least two of R², R³, R⁴and R⁵ are attached, the ring may be five-, six-, seven-membered ring. It may alternatively be a ten-, eleven-, twelve-, or thirteen-membered ring or even an eighteen-, nineteen- or twenty-membered ring.

The substituent for the lactone ring may, in particular, comprise a C₁₋₈, C₁₋₆ or C₁₋₄ linear or branched chain alkyl group such as described above. Alternatively, it may comprise a C₁₋₈, C₁₋₆ or C₁₋₄ alkenyl group and, in particular, an -CH(CH₃)CH₂ group. The substituent may also comprise a C₁₋₆ cyclic alkyl group, for example, C₁₋₄ cyclic alkyl group.

In some embodiments, a substituent may define a ring in which one or more alkylene groups (—CH₂—), for example, two, three or four, are attached to one or more carbon atoms within the carbocyclic ring. The alkylene groups may be substituted by one or more of a C₁₋₅ alkyl group, such as methyl, or by one or more further unsubstituted or C₁₋₅ alkyl substituted alkylene groups forming one or more of an additional five- or six-membered carbocyclic ring.

In particular embodiments of the uses, consumer products and methods of the present disclosure R¹ is a hydrogen atom; a C₁₋₃ linear or branched chain alkyl group; or a C₅₋₁₀ cyclic alkene group, optionally substituted with one or more of a substituent comprising a C₁₋₃ linear or branched chain alkyl or alkene group; and R², R³, R⁴ are independently a hydrogen atom; a C₁₋₅ linear or branched chain alkyl group; or a C₅₋₁₀ cyclic alkene group, optionally substituted with one or more of a substituent comprising a C₁₋₃ linear or branched chain alkyl or alkene group; or at least two of R¹, R², R³ and R⁴ are bonded together to form a saturated C₅₋₂₀ carbocyclic ring including the carbon atoms to which the at least two of R¹, R², R³, and R⁴ are attached, the ring being optionally substituted with one or more of a substituent comprising a C₁₋₅ linear or branched chain alkyl or alkene group; and the remainder of R¹, R², R³ and R⁴ are as described above.

In certain embodiments of the uses, consumer products and methods of the present disclosure, the α-selenide or β-selenide derivative is a derivative of one of the following compounds: a-damascone, β-damascone, Y-damascone, δ-damascone, a-ionone, β-ionone, Y-ionone, δ-ionone, β-damascenone, 3-methyl-5-propyl-2-cyclohexen-1-one, 1(6),8-P-menthadien-2-one, 2,5-dimethyl-5-phenyl-1-hexenone, 1-(5,5-dimethyl-1-cyclohexen-1-yl)-4-penten-1-one, 8-methyl a-ionone, 10-methyl a-ionone, 2-octenal, 1-(2,2,3,6-tetramethyl-1-cyclohexyl)-2-buten-1-one, 2-cyclo-penta-decen-1-one, nootkatone, 4-(2,2,3,6-tetramethyl-1-cyclohexyl)-3-buten-2-one, ethyl 2-octenoate, cinnamic aldehyde, methyl 2-nonenoate, 2,6,6-trimethyl-bicyclo[3.1.1.]heptane-3-spiro-2′-cyclohexen-4′-one, 2,4-decadien-oate, ethyl 2,4-undecadienenotate, methyl 5,9-dimethyl-2,4,8-decatrienoate, trans-2-hexenal and carvone.

In preferred embodiments of the uses, consumer products and methods of the present disclosure the selenide derivative has chemical formula:

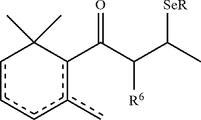

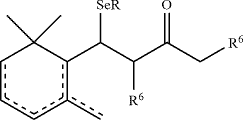

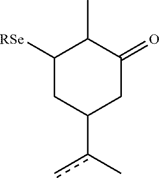

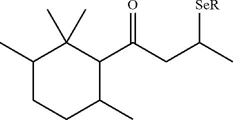

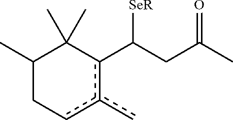

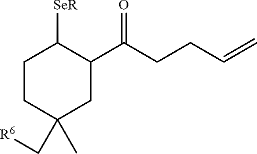

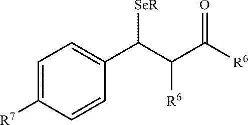

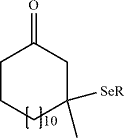

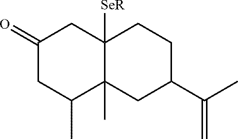

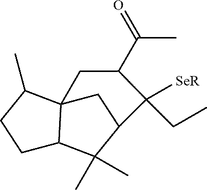

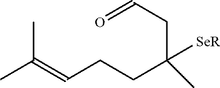

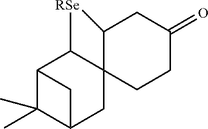

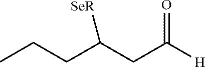

wherein the dotted line indicates the presence of one or two double carbon-carbon bonds between any adjacent pairs of the carbon atoms along which the dotted line extends;
R⁶ is a hydrogen atom or a methyl group; and
R⁷ is a hydrogen atom, a hydroxy group, a methoxy group or a linear or branched chain C₁-C₄ alkyl group.

In such preferred uses, consumer products and methods, R is preferably a C₅₋₁₆ linear alkyl group, more preferably R is a C₁₀₋₁₄ linear alkyl group, and most preferably R is a C₁₂ linear alkyl group.

In particularly preferred embodiments of the uses, consumer products and methods of the present disclosure the selenide derivative has chemical formula:

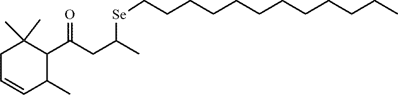 [SMILES: CCCCCCCCCCCC[Se]C(C)CC(=O)C1C(C)C=CCC1(C)C]

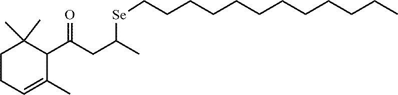 [SMILES: CCCCCCCCCCCC[Se]C(C)CC(=O)C1C(C)=CCCC1(C)C]

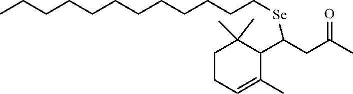 [SMILES: CCCCCCCCCCCC[Se]C(CC(C)=O)C1C(C)=CCCC1(C)C]

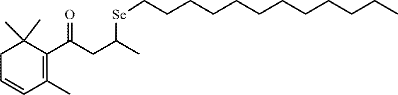 [SMILES: CCCCCCCCCCCC[Se]C(C)CC(=O)C1=C(C)C=CCC1(C)C]

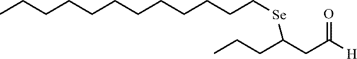 [SMILES: CCCCCCCCCCCC[Se]C(CC=O)CCC]

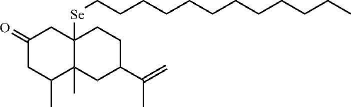 [SMILES: C=C(C)C1CCC2([Se]CCCCCCCCCCCC)CC(=O)CC(C)C2(C)C1]

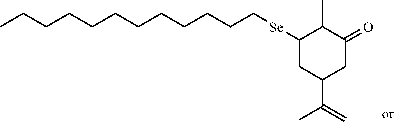

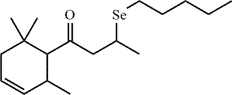 [SMILES: CCCCC[Se]C(C)CC(=O)C1C(C)C=CCC1(C)C]

The selenide derivatives show a surprising stability in appropriate storage and can deliver the fragrance compound comprising an a,β-unsaturated aldehydes, ketone, ester, lactone or carboxylic acid moiety in ambient environment with superior staying power (or tenacity) on all manner of surfaces, especially on textiles, as compared with the corresponding β-sulfide derivatives.

The consumer products of this disclosure include a fragrance composition such as a fine perfume, a cologne, an after-shave or a perfume intended for use in a consumer product.

These consumer products may simply comprise a fragrance composition comprising one or more of the selenide derivatives and a solvent commonly used in perfumery. Suitable solvents include, but are not limited to, one or more of ethanol, water, dipropylene glycol, diethyl phthalate, isopropyl myristate, benzyl benzoate, 2-(2-ethoxy)-1-ethanol or ethyl citrate, a terpene such as limonene, a glycol ether or a glycol ether ester. Alternatively, the fragrance composition may further comprise one or more fragrance compounds (not being selenide derivatives).

The fragrance composition may also contain other fragrance compounds, such as preservatives, which are commonly employed in perfumery.

The number of ingredients (including fragrance compounds and selenide derivatives) in the fragrance composition may vary between 1 and 80, for example, between 5 or 10 and 20, 30 or 50. Preferably, the number of ingredients will not exceed 10 or 20.

In any case, the amount of the selenide derivative in the fragrance composition may be between 0.1% and 10%, for example, between 0.1% and 5.0% or 3.0% and, in particular, about 0.1% to 1.0% of the fragrance composition.

Other consumer products of this disclosure include, but are not limited to, a liquid or solid detergent, a fabric softener, a fabric refresher, an ironing water, a paper, a bleach, a shampoo, a coloring preparation, a hair spray, a vanishing cream, a deodorant or anti-perspirant, a soap, a shower or bathroom mousse, an oil or gel, a hygiene product, an air freshener, or a wipe.

In these consumer products, the use may comprise the inclusion or addition of a fragrance composition as described above. Further, the fragrance composition (or selenide derivatives) may be encapsulated.

Suitable encapsulants include, but are not limited to, those mentioned in EP 1 964 554 A2.

The consumer product may, in particular, be a fabric detergent, a fabric softener or a fabric refresher. Suitable fabric detergent, fabric softener and fabric refresher compositions are, for example, described in Ullman’s Encyclopedia of Industrial Chemistry, Volume A8 (1987), pages 315 to 448 and Volume A25 (1994), pages 747 to 817 as well as in “Advanced Cleaning Product Formulations” by Flick et al., Noye Publications, Park Ridge, New Jersey, United States (1989); “Surfactant Science Series” by Showell et al., Volume 71; Powdered Detergents, Marcel Dekker, New York, United States, 1988; and Proceedings of the World Conference on Detergents (4^{th}, 1998, Montreux, Switzerland, ACOS print).

It appears that at least some of the selenide derivatives of the present disclosure are not described in the chemical literature - even as synthetic intermediates for fragrance compounds comprising a,β-unsaturated aldehyde, ketone, ester, lactone or carboxylic acid moiety.

Accordingly, in a fourth aspect, the present disclosure provides a selenide compound having the formula:

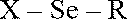

wherein X is a group generating a fragrance compound comprising an a,β-unsaturated aldehyde, ketone, ester, lactone or carboxylic acid moiety by elimination of an ambient oxidation product of the group Se — R; and wherein R is a C₅₋₁₆ linear, cyclic or branched chain alkyl group, and preferably a C₁₀₋₁₄ linear alkyl group, more preferably a C₁₂ linear alkyl group.

The selenide compound may be an a-selenide or a β-selenide derivative of the fragrance compound having the chemical formula:

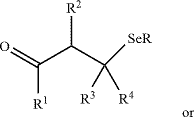

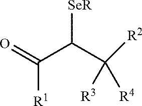

wherein R¹ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group or a C₁₋₁₂ alkoxy group; or
R¹ is a group — OR⁵, wherein R⁵ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group, or a C₁₋₁₂ alkoxy group; and
R², R³, R⁴ are independently a hydrogen atom; a C₁₋₂₀ linear or branched chain alkyl, alkenyl alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group optionally substituted with one or more of a substituent comprising a C₁₋₁₂ linear or branched chain alkyl group, a hydroxy or a C₁₋₁₂ alkoxy group; or
at least two of R¹, R², R³ and R⁴ are bonded together to form a saturated or unsaturated C₅₋₂₀ carbocyclic ring including the carbon atoms to which the at least two of R¹, R², R³, and R⁴ are attached, the ring being optionally substituted with one or more of a substituent comprising a C₁₋₁₂ linear, cyclic or branched chain or alkene alkyl group; and the remainder of R¹, R², R³ and R⁴ are as described above; or
when R¹ is the group —OR⁵, R⁵ and one of R², R³ and R⁴ are bonded together to form a saturated or unsaturated C₄₋₁₉ lactone ring including the carbon atoms to which the at least two of R², R³, R⁴and R⁵ are attached, the ring being optionally substituted with one or more of a substituent comprising a C₁₋₁₂ linear, cyclic or branched chain alkyl or alkene group; and the remainder of R², R³, R⁴ and R⁵ are as described above.

In preferred embodiments of the present disclosure, R¹ is a hydrogen atom; a C₁₋₃ linear or branched chain alkyl group; or a C₅₋₁₀ cyclic alkene group, optionally substituted with one or more of a substituent comprising a C₁₋₃ linear or branched chain alkyl or alkene group; and

R², R³, R⁴ are independently a hydrogen atom; a C₁₋₅ linear or branched chain alkyl group; or a C₅₋₁₀ cyclic alkene group, optionally substituted with one or more of a substituent comprising a C₁₋₃ linear or branched chain alkyl or alkene group; or at least two of R¹, R², R³ and R⁴ are bonded together to form a saturated C₅₋₂₀ carbocyclic ring including the carbon atoms to which the at least two of R¹, R², R³, and R⁴ are attached, the ring being optionally substituted with one or more of a substituent comprising a C₁₋₅ linear or branched chain alkyl or alkene group; and the remainder of R¹, R², R³ and R⁴ are as described above.

The selenide compound may, in particular, be an α-selenide or a β-selenide derivative of a-damascone, β-damascone, Y-damascone, or δ-damascone, α-ionone, β-ionone, Y-ionone, 5-ionone, β-damascenone, 3-methyl-5-propyl-2-cyclohexen-1-one, 1(6),8-P-menthadien-2-one, 2,5-dimethyl-5-phenyl-1-hexenone, 1-(5,5-dimethyl-1-cyclohexen-1-yl)-4-penten-1-one, 8-methyl α-ionone, 10-methyl a-ionone, 2-octenal, 1-(2,2,3,6-tetramethyl-1-cyclohexyl)-2-buten-1-one, 2-cyclo-penta-decen-1-one, nootkatone, 4-(2,2,3,6-tetramethyl-1-cyclohexyl)-3-buten-2-one, ethyl 2-octenoate, cinnamic aldehyde, methyl 2-nonenoate, 2,6,6-trimethyl-bicyclo[3.1.1.]heptane-3-spiro-2′-cyclo-hexen-4′-one, 2,4-decadien-oate, ethyl 2,4-undecadienenotate, methyl 5,9-dimethyl-2,4,8-decatrienoate, trans-2-hexenal and carvone.

The selenide compound may, in particular, have chemical formula:

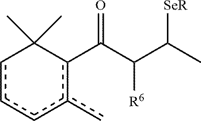

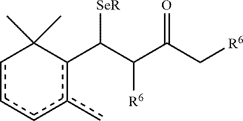

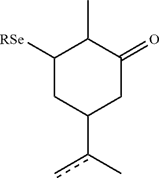

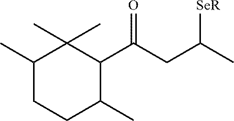

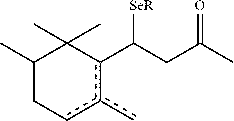

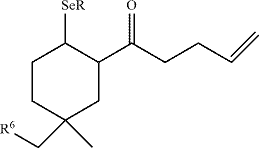

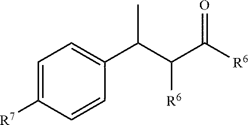

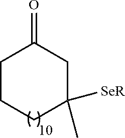

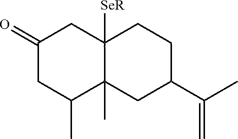

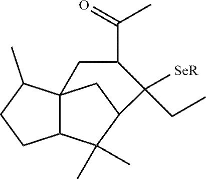

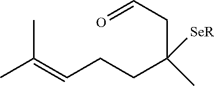

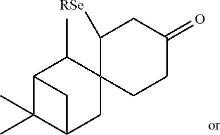

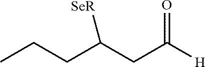

wherein the dotted line indicates the presence of one or two double carbon-carbon bonds between any adjacent pairs of the carbon atoms along which the dotted line extends;
R⁶ is a hydrogen atom or a methyl group; and R⁷ is a hydrogen atom, a hydroxy group, a methoxy group or a linear or branched chain C₁-C₄
Other embodiments (relating, for example, to corresponding the a-selenides) in this aspect of the present disclosure will be apparent from the embodiments described in relation to the first, second and third aspects of the present disclosure.

In a fifth aspect, the present disclosure provides a method for the preparation of a selenide derivative of a fragrance compound comprising an a,β-unsaturated aldehyde moiety, an a,β-unsaturated ketone moiety, an a,β-unsaturated ester moiety, an a,β-unsaturated lactone moiety or an a,β-unsaturated carboxylic acid moiety having the formula

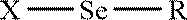

wherein X is a group generating a fragrance compound comprising an a,β-unsaturated aldehyde, an a,β-unsaturated ketone, an a,β-unsaturated ester, an a,β-unsaturated lactone or an a,β-unsaturated carboxylic acid by elimination of an ambient oxidation product of the group Se — R; and wherein R is a C₅₋₁₆ linear, cyclic or branched chain alkyl group, preferably wherein R is a C₁₀₋₁₄ linear alkyl group, more preferably a C₁₂ alkyl group; the method comprising reacting the fragrance compound with an alkyl, aryl or heterocyclic selenide ion formed by treatment of a dialkyl, diaryl or diheterocyclic diselenide with borohydride; wherein the dialkyl, diaryl or diheterocyclic diselenide has chemical formula:

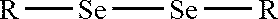

The method may, in particular, comprise Michael addition of alkyl, aryl or heterocyclic selenide ion to the fragrance compound as mentioned above.

In preferred embodiments of the method of preparation of the present disclosure the fragrance compound comprising the a,β-unsaturated aldehyde, ketone, ester, lactone or carboxylic acid moiety may have a chemical formula:

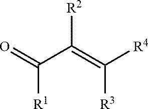

wherein R¹ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group or a C₁₋₁₂ alkoxy group; or
R¹ is a group — OR⁵, wherein R⁵ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group, or a C₁₋₁₂ alkoxy group; and
R², R³, R⁴ are independently a hydrogen atom; a C₁₋₂₀ linear or branched chain alkyl, alkenyl alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group optionally substituted with one or more of a substituent comprising a C₁₋₁₂ linear or branched chain alkyl group, a hydroxy or a C₁₋₁₂ alkoxy group; or
at least two of R¹, R², R³ and R⁴ are bonded together to form a saturated or unsaturated C₅₋₂₀ carbocyclic ring including the carbon atoms to which the at least two of R¹, R², R³, and R⁴ are attached, the ring being optionally substituted with one or more of a substituent comprising a C₁₋₁₂ linear, cyclic or branched chain alkyl or alkene group; and the remainder of R¹, R², R³ and R⁴ are as described above; or
when R¹ is the group —OR⁵, R⁵ and one of R², R³ and R⁴ are bonded together to form a saturated or unsaturated C₄₋₁₉ lactone ring including the carbon atoms to which the at least two of R², R³, R⁴and R⁵ are attached, the ring being optionally substituted with one or more of a substituent comprising a C₁₋₁₂ linear, cyclic or branched chain alkyl or alkene group; and the remainder of R², R³, R⁴ and R⁵ are as described above.

In certain embodiments, the fragrance compound comprising α,β-unsaturated aldehyde, ketone, ester, lactone or carboxylic acid is β-damascone, _{Y}-damascone, or δ-damascone, a-ionone, β-ionone, _{Y}-ionone, 5-ionone, β-damascenone, 3-methyl-5-propyl-2-cyclohexen-1-one, 1(6),8-P-menthadien-2-one, 2,5-dimethyl-5-phenyl-1-hexenone, 1-(5,5-dimethyl-1-cyclohexen-1-yl)-4-penten-1-one, 8-methyl a-ionone, 10-methyl a-ionone, 2-octenal, 1-(2,2,3,6-tetramethyl-1-cyclohexyl)-2-buten-1-one, 2-cyclo-penta-decen-1-one, nootkatone, 4-(2,2,3,6-tetramethyl-1-cyclohexyl)-3-buten-2-one, ethyl 2-octenoate, cinnamic aldehyde, 2,6,6-trimethyl-bicyclo[3.1.1.]heptane-3-spiro-2′-cyclo-hexen-4′-one, 2,4-decadien-oate, methyl 2-nonenoate, ethyl 2,4-undecadienenotate, methyl 5,9-dimethyl-2,4,8-decatrienoate, trans-2-hexenal and carvone.

Other embodiments in this aspect of the present disclosure will be apparent from the embodiments described in relation to the first, second, third and fourth aspects of the present disclosure.

In a sixth aspect, the present disclosure provides a method for the preparation of an α-selenide derivative of a fragrance compound comprising an α,β-unsaturated aldehyde moiety, an α,β-unsaturated ketone moiety, an α,β-unsaturated ester moiety, an α,β-unsaturated lactone moiety or an α,β-unsaturated carboxylic acid moiety.

In one embodiment, the method comprises reacting the fragrance compound with an alkyl, aryl or heterocyclic selenol formed by treatment of a dialkyl diselenide, diaryl diselenide or diheterocyclic selenide with tri-n-butyl tin hydride and a radical initiator such as azobisiso-butyronitrile (AIBN).

The method may, in particular, comprise Michael addition of hydrogen atom to the fragrance compound followed by quenching of the radical addition product as mentioned above.

In certain embodiments, the selenol has chemical formula:

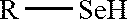

wherein R is a C₅₋₁₆ linear, cyclic or branched chain alkyl group, preferably wherein R is a C₁₀₋₁₄ linear alkyl group, more preferably a C₁₂ linear alkyl group.

In another embodiment, the method comprises hydrogenating the fragrance compound (for example, by reacting with hydrogen in the presence of a catalyst, such as platinum), treating the hydrogenated fragrance compound with a strong base (for example, n-BuLi or NH₃) and quenching the resultant anion with an alkyl (or other) selenyl halide, such as an alkylselenyl chloride.

Embodiments in this aspect of the present disclosure will be apparent from the embodiments described in relation to the first, second, third, fourth and fifth aspects of the present disclosure.

In a seventh aspect, the present disclosure provides a method of prolonging the characteristic fragrance note of a fragrance composition comprising a fragrance compound comprising an α,β-unsaturated aldehyde moiety, an α,β-unsaturated ketone moiety, an α,β-unsaturated ester moiety, an α,β-unsaturated lactone moiety or an α,β-unsaturated carboxylic acid moiety by providing that the fragrance composition comprises a selenide derivative of the fragrance compound in place, of or in addition, to the fragrance compound in the fragrance composition.

Embodiments in this aspect of the present disclosure will be apparent from the embodiments described in relation to the first to sixth aspects of the present disclosure.

In an eighth aspect, the present disclosure provides for use of an a-sulfide (thioether) derivative of a fragrance compound comprising an α,β-unsaturated aldehyde moiety, an α,β-unsaturated ketone moiety, an α,β-unsaturated ester moiety, an α,β-unsaturated lactone moiety or an α,β-unsaturated carboxylic acid moiety in a consumer product.

In further aspects, the present disclosure also provides a-sulfide derivatives of the fragrance compound as well as a method for their preparation and a consumer product comprising an a-sulfide derivative of the fragrance compound.

It also provides a method of perfuming an article or surface comprising treating the article or surface with a fragrance composition comprising an a-sulfide derivative of the fragrance compound as well as a method of prolonging the characteristic fragrance note of a fragrance composition comprising the fragrance compound by providing that the fragrance composition comprises an a-sulfide derivative of the fragrance compound.

Embodiments in these aspects of the present disclosure will be apparent from the embodiments described in relation to the first to seventh aspects of the present disclosure.

The present disclosure will now be described in more detail by way of Example only with reference to the following Examples and the accompanying drawings in which:

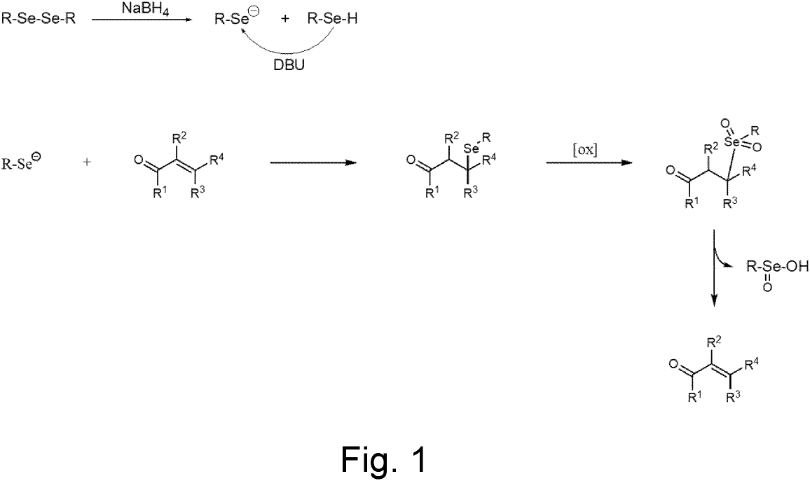

FIG. 1 is a scheme generally outlining the formation of a β- selenide derivative of an a,β-unsaturated aldehyde, ketone, ester, lactone or carboxylic acid and the regeneration of the a,β-unsaturated aldehyde, ketone, ester, lactone or carboxylic acid in ambient environment;

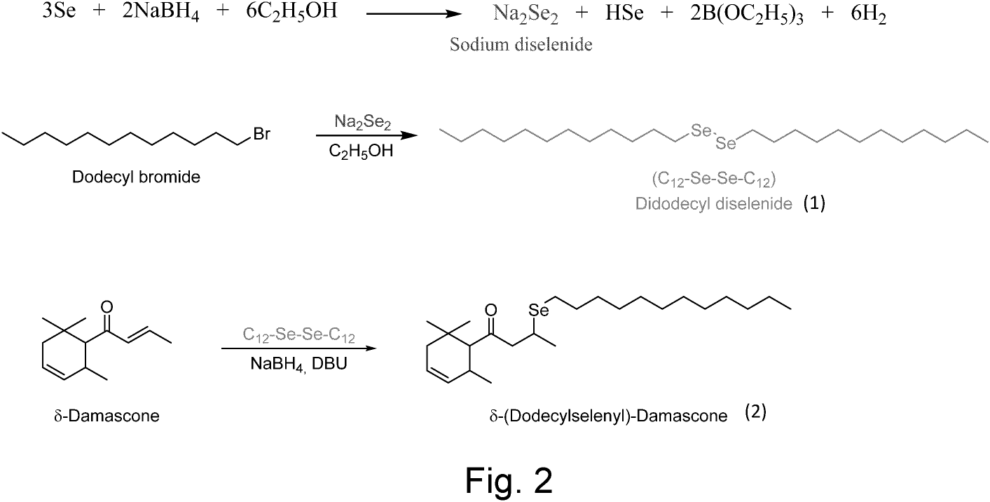

FIG. 2 is a scheme outlining the preparation of a β-selenide derivative of a fragrance compound according to one embodiment of the present disclosure; and

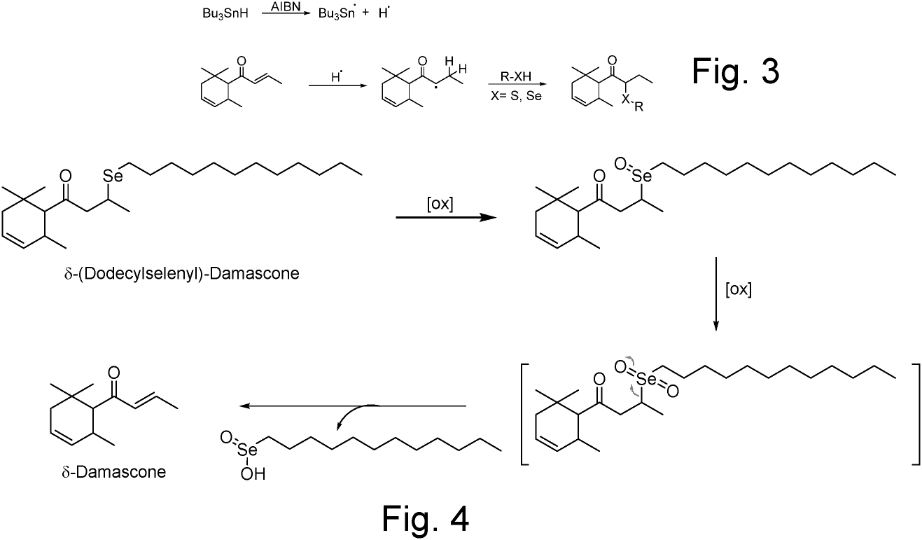

FIG. 3 is a scheme outlining the preparation of an α-selenide derivative of a fragrance compound according to one embodiment of the present disclosure; and

FIG. 4 is a scheme outlining the regeneration in ambient atmosphere of a fragrance compound according to one embodiment of the present disclosure.

Referring now to FIG. 1, there is shown a reaction scheme generally illustrating the generation of alkyl selenide ion (RSe⁻) from a dialkyl diselenide by treatment with sodium borohydride (NaBH₄) and subsequent reaction (by conjugate addition) of the alkyl selenide ion with an a, β-unsaturated aldehyde, ketone, or ester to form a selenide derivative (at the β position) of the a, β-unsaturated aldehyde, ketone, or ester.

A suitable method for carrying out the reaction is described by Gelson Perin, Elton L. Borges, Paloma C. Rosa, Patrick N. Carvalho, and Eder João Lenardao in Tetrahedron Letters 54 (2013) 1718-1721.

Note that 1,8-diazabicyclo[5.4.0]undec-7-ene (DBU) may be used in addition to sodium borohydride in order to convert the alkyl selenol (RSeH), a reaction byproduct of sodium borohydride with the dialkyl diselenide, to generate further alkyl selenide ion (RSe⁻).

The reaction scheme also shows the decomposition of the selenide derivative by oxidation and subsequent elimination of the oxidation product to regenerate the a, β-unsaturated aldehyde, ketone, or ester.

Note that the decomposition may proceed by oxidation to selenoxide or selenone and elimination of either an alkyl seleninic acid or an alkyl selenenic acid.

The dialkyl diselenide can, for example, be prepared in a one-pot reaction comprising treatment of selenium with sodium borohydride to generate disodium diselenide and subsequent reaction with an alkyl, aryl or heterocyclic bromide.

One such method for preparing the dialkyl diselenide (as well as diaryl diselenide or diheterocyclic diselenide) is described by Daniel L. Klayman and T. Scott Griffin in J. Am. Chem. Soc. 1973, 95, 1, 197-199.

Referring now to FIG. 2, there is shown a synthesis of didodecyl diselenide and the preparation of the dodecyl selenide derivative of δ-damascone (δ-(dodecylselenyl)damascone) in accordance with one embodiment of the present disclosure.

As may be seen, the treatment of didodecyl diselenide with sodium borohydride and DBU in the presence of δ-damascone results in conjugate (Michael) addition at the β-position of the α,β-unsaturated ketone moiety.

Referring now to FIG. 3, there is shown a reaction scheme generally illustrating the generation of hydrogen atom from tri-n-butyl tin hydride and its subsequent addition to δ-damascone at the β-position of the α,β-unsaturated ketone moiety. The addition leads to a comparatively stable radical at the a-position of the ketone moiety which is quenched by the addition of an alkyl (or other) selenol so that an alkyl (or other) selenide group is formed at the a-position.

Note that the radical may also be quenched by an alkyl thiol and that the a-sulfide derivative so obtained may be of some value as compared to the β-sulfide derivative for the controlled release of δ-damascone.

Referring now to FIG. 4, there is shown a reaction scheme generally describing the decomposition of the dodecyl selenide derivative of δ-damascone in ambient environment to regenerate δ-damascone.

The decomposition of 5-(dodecylselenyl)damascone in ambient is thought to proceed through oxidation by air to selenoxide and thence selenone and elimination of an alkyl seleninic acid.

However, as mentioned above, the decomposition may also proceed through oxidation to selenoxide and elimination of an alkyl selenenic acid.

The following examples describe in detail the preparation of δ-(dodecylselenyl)damascone, a fragrance composition comprising δ-(dodecylselenyl)damascone, and a fabric softener comprising the fragrance composition.

### Example 1 - Preparation of Dodecyl Selenide Derivative of δ-Damascone Preparation of Didodecyl Diselenide

Didodecyl selenide was prepared in line with the method described by Daniel L. Klayman and T. Scott Griffin in J. Am. Chem. Soc. 1973, 95, 1, 197-199.

To a suspension of selenium (2.5 g; 31.6 mmol) in ethanol (30 ml), 2.4 g (63.3 mmol) of NaBH₄ were added and the mixture stirred at room temperature. Considerable foaming (due to H₂) occurred immediately in the vigorous reaction and the selenium was consumed in less than 20 min. Then, others 2.5 g (31.6 mmol) of selenium were added and the mixture was stirred 10 min more. The formation of sodium diselenide was confirmed by TLC. Then, 15.7 g (63.3 mmol) of dodecyl bromide were added and the mixture was stirred at room temperature for 24 hr. The reaction progress was followed by TLC. Once the reaction was completed, the solvent was evaporated and the crude extracted with hexane (20 ml), filtered and dried over Na₂SO₄ and the solvent evaporated under reduced pressure. Yield of crude product: 17.2 g (83%).

### Preparation of 3-(Dodecylselenyl)-1-(2,6,6-Trimethyl-3-Cyclohexen-1-yl)-1-Butanone

The β-dodecyl selenide derivative of δ-damascone was prepared in line with the method described by Gelson Perin, Elton L. Borges, Paloma C. Rosa, Patrick N. Carvalho, and Eder João Lenardao in Tetrahedron Letters 54 (2013) 1718-1721.

To a mixture of didodecyl diselenide prepared as above (5 g; 10.07 mmol) in THF (30 ml), under nitrogen atmosphere, δ-damascone (5.8 g; 30.21 mmol), NaBH₄ (0.38 g; 10.07 mmol) and DBU (1.53 g; 10.07 mmol) were added and the mixture was stirred at room temperature, following the reaction progress by TLC. After 2 h of reaction, the solution was cooled to room temperature, diluted with ethyl acetate (20 ml) and washed with water (3 x 10 ml). The organic phase was separated, dried over Na₂SO₄ and the solvent evaporated under reduced pressure. Yield of crude product: 10.1 g (93.5 %).

The β-dodecyl selenide derivative was isolated by column chromatography using hexane or hexane/ethyl acetate as eluent - and characterized as follows: 1H-NMR: 0.80-0.90 (m, 6H); 0.90-1.05 (m, 6 H); 1.25 (m, 18 H); 1.42 (d, 3 H); 1.68 (m, 2 H); 1.89 (m, 1 H); 1.96 (dd, 1 H); 2.22 (dd, 1 H); 2.58-3.07 (m, 4 H); 3.42 (m, 2 H); 5.47 (m, 1 H); 5.56 (m, 1 H). 13C-NMR: 213.6 (Cq); 132.3 (CH); 124.6 (CH); 63.5 (CH); 56.1 (CH₂); 42.6 (CH2); 33.5 (CH); 32.2 (CH₃); 30.8-29.2 (Several CH₂); 27.2 (CH); 24.6 (Cq); 22.6 (CH₃); 20.5 (CH₃); 19.8 (CH₃); 14.8 (CH₃). 77Se—NMR— 286.77 ppm MS-GM Spectrometry:

$$\left[\text{M+1}\right] = 442$$

### Example 2 - Fragrance Compositions

A fragrance composition (hereinafter designated “Example Composition 1”) was prepared by adding the β-dodecyl selenide derivative of δ-damascone (of Example 1) to a standard fragrance base at a concentration of 5% w/v. The composition of the Example Composition 1 is shown in Table 1 (the β-dodecyl selenide derivative of δ-damascone is labelled “TEST COMPOUND”.

Additional fragrance compositions were prepared by replacing the β-dodecyl selenide derivative of δ-damascone in the composition of Table 1 of by 5% w/v δ-damascone (hereinafter “δ-damascone (-)”) and 5% w/v of the β-dodecyl sulfide derivative of δ-damascone (prepared as described in WO 03/049666 A2; hereinafter “competitor (S)”).

TABLE 1

| Compound | % w/v | CAS N° |
|---|---|---|
| GALAXOLIDE IPM MUI | 9.00 | 1222-05-5 |
| CYCLOVERDYL ACETATE MUI | 8.00 | 2500-83-6 |
| LILESTRALIS MUI | 8.40 | 80-54-6 |
| CITRONELLOL MUI | 7.80 | 106-22-9 |
| ROSE CRYSTALS MUI | 7.00 | 90-17-5 |
| TERPINEOL 95 MUI | 6.70 | 98-55-5 |
| ISOBORNYL ACETATE MUI | 6.30 | 125-12-2 |
| DIPROPYLENE GLYCOL MUI | 5.70 | 25265-71-8 |
| ALLYL PHENOXYACETATE | 4.60 | 7493-74-5 |
| DIPHENYL OXIDE MUI | 4.45 | 101-84-8 |
| DIHYDROTERPINYL ACETATE MUI | 3.70 | 58985-18-5 |
| BENZYL ACETATE FG MUI | 3.36 | 140-11-4 |
| TERPINEOL DBCH PERFUMERY MUI | 2.80 | 98-55-5 |
| P-TERBUTYLCYCLOHEXYL ACETATE MUI | 2.61 | 32210-23-4 |
| DIMETHYL OCTANOL / TETRAHYDROGERANIOL MUI | 2.50 | 106-21-8 |
| LINALOOL MUI | 2.20 | 78-70-6 |
| LAVANDIN GROSSO OIL MUI | 1.60 | 8022-15-9 |
| PHENYLETHYL ACETATE K MUI | 1.40 | 103-45-7 |
| ALDEHYDE C14 / GAMMA-UNDECALACTONE FG MUI | 1.00 | 104-67-6 |
| PHENYLPROPYL ALCOHOL MUI | 1.00 | 122-97-4 |
| GERANIOL EXTRA 97% MUI | 0.90 | 106-24-1 |
| NEROL EXTRA FG MUI | 0.90 | 106-25-2 |
| METHYL IONONE MUI | 0.60 | 1335-46-2 |
| CITRONELLYL ACETATE MUI | 0.50 | 150-84-5 |
| LINALYL ACETATE MUI | 0.50 | 115-95-7 |
| PHENETHYL ALCOHOL MUI | 0.20 | 60-12-8 |
| BUTYLATED HYDROXYTOLUENE MUI | 0.10 | 128-37-0 |
| MYRCENATE MUI | 0.20 | 25225-08-5 |
| EUCALYPTOL NATURAL FG K MUI | 0.10 | 470-82-6 |
| DIMETHYL MYRCETONE MUI | 0.10 | 54464-57-2 |
| L-BORNEOL CRYSTALS 64% PURITY MUI | 0.10 | 464-45-9 |
| DIHYDROMYRCENOL K MUI | 0.10 | 18479-58-8 |
| CAMPHOR 96% SYNTH MUI | 0.10 | 76-22-2 |
| GURJUM BALSAM MUI | 0.10 | 8030-55-5 |
| KOAVONE MUI | 0.10 | 81786-73-4 |
| GERANIUM OIL, EGYPT FG MUI | 0.10 | 8000-46-2 |
| COUMARIN MUI | 0.10 | 91-64-5 |
| P-METHYLACETOPHENONE FG MUI | 0.02 | 122-00-9 |
| METHYL HEXYL CETONE FG MUI | 0.02 | 111-13-7 |
| TEST COMPOUND | 5.00 | n/a |
| CITRONELLAL MUI | 0.01 | 106-23-0 |
| HEXYL ACETATE FG K MUI | 0.01 | 142-92-7 |
| ETHYL AMYL CETONE FG MUI | 0.01 | 106-68-3 |
| CITRONELLYL FORMATE FG MUI | 0.01 | 105-85-1 |
| | 100.00 | |

### Example 3 - Performance of Example Composition 1 in a Fabric Softener

The performance of Example Composition 1 (of Example 2) in a standard fabric softener base (see Example 4) was evaluated in accordance with an industry standard panel evaluation test. The additional fragrance compositions comprising δ-damascone (δ-damascone (-)) and the β-dodecyl sulfide derivative of δ-damascone (competitor (S)) were also evaluated in the same test.

The fabric softeners were prepared by mixing the respective fragrance compositions into the standard fabric softener composition. The procedure is illustrated in Example 4 (the standard fabric softener base being everything other than the fragrance composition).

The panel was made up of seven expert professionals (perfumers and evaluators) and the evaluation performed (in a room substantially free from ambient odors) on the fabric softener and samples of fabrics of similar size washed (and dried) with the fabric softeners at the same time.

Each of the fabric softeners and samples (not more than five at any one time) was labeled with a random code having a correspondence with a fragrance composition not known to the panel.

The panellists were asked to rate liking and strength of the fragrance softener and the fabric samples according to the following key:

| | |
|---|---|
| Liking: | Strength: |
| 1. Disliked | 1. Very weak |
| 2. Neither Liked nor disliked | 2. Weak |
| 3. Fair | 3. Medium Strength |
| 4. Good | 4. Strong |
| 5. Very good | 5. Very strong |

Three different fabrics types (100% cotton, combination of 60% cotton and 40% polyester, 100% polyester (comprising a mixture of contaminated and uncontaminated fabrics providing half-load ∼ 2 kg) were washed with fabric softener in respective washing machines (all the same brand and model). The fabric softeners and the washed fabrics were presented for evaluation by the panel in accordance with the following procedure:

### Phase 1: Wet Fabrics

The fabric softener was dispensed from a compartment of the washing machine on commencement of a wash cycle at room temperate which lasted 30 minutes. After the completion of the wash cycle the just washed fabric samples were evaluated as described above.

### Phase 2: Fabric Softener

10 g of each fabric softener was placed in a respective wide mouth glass jar and the neat fabric softener evaluated as described above.

### Phase 3: Dry Fabrics

The washed fabric samples (from phase 1) were hung up on respective clothes lines to dry (at room temperature) and left there during a period of ten days. The dried fabrics were evaluated as described above every two days up to 10 days.

The averaged results of the evaluation by the panel for the just washed and dried fabric samples are summarized in Table 2.

As may be seen from Table 2, the performance of Example Composition 1 in the fabric softener is from the outset generally superior to that of Competitor (S) in liking, and is superior in duration to both Competitor (S) and δ-damascone.

Without wishing to be bound by theory, it is thought that the superiority of the β-selenide is because the oxidation of selenide in ambient environment proceeds to a greater extent of completion as compared to the oxidation of sulphide on account of the accessibility of the significantly larger selenium atom as compared to sulphur atom.

TABLE 2

| Period | Sample | Averaged Liking | Averaged Strength |
|---|---|---|---|
| 0 Days | | | |
| | Competitor (S) | 3.31 | 3.36 |
| | Example Composition 1 | 3.44 | 3.20 |
| | δ-Damascone (-) | 3.46 | 3.11 |

| 2 Days | | | |
|---|---|---|---|
| | Competitor (S) | 3.37 | 3.33 |
| | Example Composition 1 | 3.51 | 3.31 |
| | δ-Damascone (-) | 2.50 | 1.69 |
| 3 Days | | | |
| | Competitor (S) | 3.64 | 3.00 |
| | Example Composition 1 | 3.62 | 3.26 |
| | δ-Damascone (-) | 2.40 | 1.40 |
| 4 Days | | | |
| | Competitor (S) | 3.21 | 2.86 |
| | Example Composition 1 | 3.40 | 2.97 |
| | δ-Damascone (-) | 1.43 | 1.07 |
| 7 Days | | | |
| | Competitor (S) | 3.06 | 2.93 |
| | Example Composition 1 | 3.36 | 3.18 |
| | δ-Damascone (-) | 1.20 | 0.75 |

### Example 4 - Fabric Softener

A fabric softener (see Table 3) comprising Example Composition 1 of Example 2 was prepared as follows:

A solution of the dye in deionised water was prepared by addition of the dye with stirring to deionised water heated to 55° C. Pre-heated (to 45° C.) dehydrogenated tallowoylethyl hydroxyethylmonium metho-sulfate was added to this solution slowly with stirring and the stirring continued until the mixture had cooled to room temperature (around 20 mins). Calcium chloride dihydrate salt diluted with a little deionised water was added to the resulting mixture and the stirring continued for further 10 minutes. Isothiazolinone preservative and Example Composition 1 was added and the resulting mixture thoroughly stirred for a further 15 minutes.

TABLE 3

| | |
|---|---|
| Ingredient (INCI Name) | % W/W |
| Dihydrogenated tallowoylethyl hydroxyethylmonium methosulfate | 16.4 |
| Methyl-4-isothiazolin 2.5% and 1,2-Benzisothiazolin-3-one 2.5% | 0.1 |
| CaCl₂.2H₂0 (15% in water) | q.s. |
| Dye (10% in water) | q.s. |
| Example Composition 1 | 1.0 |
| Deionised water | to 100 |

### Example 5 - Preparation of 3-(Dodecylselenyl)-1-(2,6,6-Trimethyl-2-Cyclohexen-1-yl)-1-Butanone

The β-dodecyl selenide derivative of a-damascone was synthesized from α-damascone and didodecyl diselenide following the procedure of Example 1.

The β-dodecyl selenide derivative was isolated by column chromatography using hexane or hexane/ethyl acetate as eluent and characterized as follows:

1H-NMR: 0.84-0.95 (m, 12 H); 1.25 (br, 18 H); 1.45 (dd, 3H); 1.55-2.25 (m, 6 H); 2.35-2.80 (m, 3 H); 2.83 (d, 1 H); 3.45 (m, 2 H); 5.59 (br, 1 H). 13C-NMR: 212.2 (Cq); 130.2 (Cq); 123.7 (CH); 64.1 (CH); 54.5 (CH2); 47.5 (CH2); 33.0-27.5 (many, Cq, CH2); 28.2 (CH3); 24.0 (CH3); 23.6 (CH2); 23.5 (CH3); 14.1 (CH3).

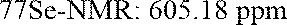

MS-GM Spectrometry:

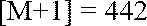

The derivative has the same fragrance profile as the original alpha-damascene with a distinctive floral fruity note with a touch of apple and blackcurrant.

### Example 6 - Preparation of 3-(Dodecylselenyl)-1-(2,6,6-Trimethyl-1,3-Cyclohexadien-1-yl)-1-Butanone

The β-dodecyl selenide derivative of β-damascenone was synthesized from β-damascenone and didodecyl diselenide following the procedure of Example 1.

The β-dodecyl selenide derivative was isolated by column chromatography using hexane or hexane/ethyl acetate as eluent and characterized as follows:

1H-NMR: 0.89 (t, 3 H); 0.97 (t, 3 H); 1.15 (t, 3 H); 1.27 (br, 18 H); 1.50 (d, 3H); 1.65 (m, 2 H); 1.72 (d, 3 H); 2.50-2.65 (dt, 2 H); 2.80-3.00 (m, 3 H); 3.52 (m, 2 H); 5.56 (d, 1 H); 5.62 (m, 1 H).

13C-NMR: 208.0 (Cq); 142.4 (Cq); 141.7 (Cq); 128.2 (CH); 127.5 (CH); 54.0 (CH2); 47.5 (CH2); 39.8 (CH2); 34.0-29.3 (many, Cq, CH2); 27.6 (CH); 26.3 (CH3); 27.0 (CH2); 22.9 (CH3); 22.8 (CH2); 19.2 (CH3); 14.3 (CH3).

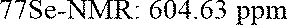

MS-GM Spectrometry:

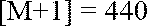

The derivative is very powerful and similar to the β-damascenone typical scent of plum, rose and tobacco.

### Example 7 - Preparation of 4-(Dodecylselenyl)-4-(2,6,6-Trimethyl-2-Cyclohexadien-1-yl)-2-Butanone

The β-dodecyl selenide derivative of a-ionone was synthesized from a-ionone and didodecyl diselenide following the procedure of Example 1.

The β-dodecyl selenide derivative was detected by Gas Cromatography and confirmed by Mass Spectroscopy.

MS-GM Spectrometry:

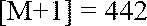

The derivative has a very similar fragrance profile to a-ionone, with a warm and woody character with a touch of violet flowers.

### Example 8 - Preparation of 3-(Dodecylselenyl)-Hexanal

The β-dodecyl selenide derivative of trans-2-hexenal was synthesized from trans-2-hexenal and didodecyl diselenide following the procedure of Example 1. The β-dodecyl selenide derivative was detected by Gas Chromatography and confirmed by Mass Spectroscopy.

MS-GM Spectrometry:

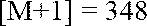

The derivative maintains the same profile, fruity green, as the trans-2-hexenal during the first 48 hours, and after that the smell changes to a pleasant grapefruit with lemon notes.

### Example 9 - Preparation of 8a-(Dodecylselenyl)-4,4a-Dimethyl-6-(Prop-1-en-2-yl)-4,4a,5,6,7,8-Hexahydro-Naphthalen-2-One

The β-dodecyl selenide derivative of nootkatone was synthesized from nootkatone and didodecyl diselenide following the procedure of Example 1. The β-dodecyl selenide derivative was detected by Gas Chromatography and confirmed by Mass Spectroscopy.

MS-GM Spectrometry:

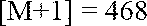

The derivative has the same fragrance profile as the original Nootkatone, with an intensive grapefruit, citrus and lightly woody fragrance.

### Example 10 - Preparation of 3-(Dodecylselenyl)-5-Isopropenyl-2-Methylcyclohexanone

The β-dodecyl selenide derivative of carvone was synthesized from carvone and didodecyl diselenide following the procedure of Example 1. The β-dodecyl selenide derivative was detected by Gas Chromatography and confirmed by Mass Spectroscopy.

MS-GM Spectrometry:

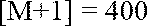

The derivative has a similar fragrance profile as the original Carvone, suit mint and spearmint leaves.

### Example 11

### Preparation of Dipentyl Diselenide

Dipentyl selenide compound was synthesized from n-pentyl bromide and selenium following the procedure of Example 1. The product was detected by Gas Chromatography and confirmed by Mass Spectroscopy.

MS-GM Spectrometry:

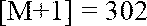

### Preparation of 3-(Pentylselenyl)-1-(2,6,6-Trimethyl-3-Cyclohexen-1-yl)-1-Butanone

The synthesis of the β-pentyl selenide derivative of δ-damascone was performed from δ-damascone and dipentyl diselenide following the procedure of Example 1. The β-pentyl selenide derivative was detected by Gas Chromatography and confirmed by Mass Spectroscopy.

1H-NMR: 0.86 (d, 3H); 0.90-1.10 (m, 9H); 1.30-1.50 (m, 4 H); 1.60-1.80 (m, 2H); 1.91 (d, 3H); 1.98 (m, 1H); 2.22 (dd, 2H); 2.48 (d, 1H); 2.55-2.72 (m, 2H); 2.80-3.05 (m, 3H); 5.40-5.60 (m, 1H); 6.23 (dd, 1H).

13C-NMR: 203.9 (Cq); 132.3 (CH); 124.2 (CH); 60.3 (CH); 55.5 (CH₂); 42.1 (CH₂); 31.5 (CH); 30.1 (CH₂); 29.9 (CH₃); 23.2 (CH₂); 22.6 (Cq); 21.1 (CH₃); 20.7 (CH₃); 18.4 (CH₃); 14.1 (CH₃).

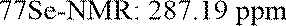

MS-GM Spectrometry:

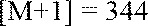

The derivative has the same fragrance profile as the original Delta Damascone with an intense Rose, floral woody and slightly citrus and herbal notes.

### Example 12 - Preparation of Example Compositions 5, 6 and 8

The α-damascone derivative of Example 5, β-damascenone derivative of Example 6 and the trans-2-hexenal derivative of Example 8 were each used to prepare fragrance compositions (hereinafter designated “Example Composition 5”, “Example Composition 6” and “Example Composition 8” respectively). The fragrance compositions were prepared by adding 5% w/v of the respective derivative to the reference fragrance composition used in Example 2, but using 5% w/v of the derivative as the “TEST COMPOUND” to replace the β-dodecyl selenide derivative of δ-damascone.

### Example 13 - Performance of Example Compositions 5, 6 and 8 in a Fabric Softener

The performances of Example Compositions 5, 6 and 8 were compared in the standard fabric softener base of Example 4. In each case, 1%w/w of the respective Example Composition was used instead of Example Composition 1.

The fabric softeners were evaluated using the procedure set out in Example 3, although the evaluation panel was made up of 9 expert professionals (perfumers and evaluators) rather than 7.

The fabric softeners were each used to wash three different fabric types, as set out in example 3, and the panellists were asked to rate liking and strength of the fabric softeners and washed fabrics using the according to the key set out in Example 3.

The average results for the evaluation by the panel of the fabric softeners comprising each Example Composition are set out in Table 4.

TABLE 4

| Sample | Averaged Liking | Averaged Strength |
|---|---|---|
| Example Composition 5 | 3.44 | 3.57 |
| Example Composition 6 | 3.63 | 3.87 |
| Example Composition 8 | 3.49 | 3.78 |

The average results for the evaluation by the panel of the just washed (wet) fabric samples and the dried fabric samples are set out in Table 5.

TABLE 5

| Period | Sample | Averaged Liking | Averaged Strength |
|---|---|---|---|
| 0 Days (wet) | | | |
| | Example Composition 5 | 3.44 | 3.34 |
| | Example Composition 6 | 3.66 | 3.67 |
| | Example Composition 8 | 3.37 | 3.63 |

| | | | |
|---|---|---|---|
| 1 Day | | | |
| | Example Composition 5 | 3.52 | 3.52 |
| | Example Composition 6 | 3.6 | 3.51 |
| | Example Composition 8 | 3.66 | 3.58 |
| | Example Composition 5 | 3.61 | 3.32 |
| | Example Composition 6 | 3.65 | 3.40 |
| | Example Composition 8 | 3.70 | 3.44 |
| | Example Composition 5 | 3.40 | 3.42 |
| | Example Composition 6 | 3.56 | 3.58 |
| | Example Composition 8 | 3.68 | 3.52 |

The Examples demonstrate the suitability of the selenide derivatives of fragrance compounds to provide for long lasting controlled release of the fragrance compound.

They also show that the selenide derivatives provide that the fragrance composition has a note which is cleaner (or clearer) than that of the corresponding sulfide derivative and also that the release of the fragrance may be longer lasting than for the corresponding sulfide derivative.

The fragrance on a fabric article treated with a fabric softener containing the selenide derivative can persist for a period of at least three days, for example at least 5 days, at least 10 days, at least 21 days, at least 35 days or even longer.

1. (canceled)

2. A consumer product comprising a selenide derivative of a fragrance compound comprising an α,β-unsaturated aldehyde moiety, an α,β -unsaturated ketone moiety, an α,β -unsaturated ester moiety, an α,β -unsaturated lactone moiety or an α,β -unsaturated carboxylic acid moiety.

3. A consumer product according to claim 2, which is a fine perfume, a cologne, an after-shave lotion, a liquid or solid detergent, a fabric softener, a fabric refresher, an ironing water, a paper, a bleach, a shampoo, a coloring preparation, a hair spray, a vanishing cream, a deodorant or anti-perspirant, a soap, a shower or bathroom mousse, an oil or gel, a hygiene product, an air freshener, or a wipe.

4. A method of perfuming an article or surface comprising treating the article or surface with a selenide derivative of a fragrance compound comprising an α,β-unsaturated aldehyde moiety, an α,β-unsaturated ketone moiety, an α, -unsaturated ester moiety, an α,β-unsaturated lactone moiety, or an α,β -unsaturated carboxylic acid moiety.

5. A consumer product according to claim 2, wherein the selenide derivative has chemical formula: 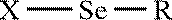
wherein X is a group generating the fragrance compound by elimination of an ambient oxidation product of the group Se — R; and wherein R is a C₁₋₂₀ linear, cyclic or branched chain alkyl group or a C₆₋₁₀ unsubstituted or substituted aryl or heteroaryl group, wherein the substituent is a C₁₋₁₂ linear, cyclic or branched chain alkyl group; preferably wherein R is a C₅₋₁₆ linear alkyl group, more preferably wherein R is a C₁₀₋₁₄ linear alkyl group, most preferably a C₁₂ linear alkyl group.

6. A, consumer product according to claim 5, wherein the selenide derivative has chemical formula: 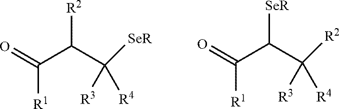
or
wherein R¹ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group or a C₁₋₁₂ alkoxy group; or
R¹ is a group — OR⁵, wherein R⁵ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group, or a C₁₋₁₂ alkoxy group; and
R², R³, R⁴ are independently a hydrogen atom; a C₁₋₂₀ linear or branched chain alkyl, alkenyl alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group optionally substituted with one or more of a substituent comprising a C₁₋₁₂ linear or branched chain alkyl group, a hydroxy or a C₁₋₁₂ alkoxy group; or
at least two of R¹, R², R³ and R⁴ are bonded together to form a saturated or unsaturated C₅₋₂₀ carbocyclic ring including the carbon atoms to which the at least two of R¹, R², R³, and R⁴ are attached, the ring being optionally substituted with one or more of a substituent comprising a C₁₋₁₂ linear, cyclic or branched chain alkyl or alkene group; or
when R¹ is the group —OR⁵, R⁵ and one of R², R³ and R⁴ are bonded together to form a saturated or unsaturated C₄₋₁₉ lactone ring including the carbon atoms to which the at least two of R², R³, R⁴ and R₅ are attached, the ring being optionally substituted with one or more of a substituent comprising a C₁₋₁₂ linear, cyclic or branched chain alkyl or alkene group ⁴ .

7. A consumer product according to claim 6, wherein R¹ is a hydrogen atom; a C₁₋₃ linear or branched chain alkyl group; or a C₅₋₁₀ cyclic alkene group, optionally substituted with one or more of a substituent comprising a C₁₋₃ linear or branched chain alkyl or alkene group; and
R², R³, R⁴ are independently a hydrogen atom; a C₁₋₅ linear or branched chain alkyl group; or a C₅₋₁₀ cyclic alkene group, optionally substituted with one or more of a substituent comprising a C₁₋₃ linear or branched chain alkyl or alkene group; or
at least two of R¹, R², R³ and R⁴ are bonded together to form a saturated C₅₋₂₀ carbocyclic ring including the carbon atoms to which the at least two of R¹, R², R³, and R⁴ are attached, the ring being optionally substituted with one or more of a substituent comprising a C₁₋₅ linear or branched chain alkyl or alkene group; and the remainder of R¹, R², R³ and R⁴ are as described above.

8. A consumer product according to claim 6, wherein the selenide derivative has chemical formula: 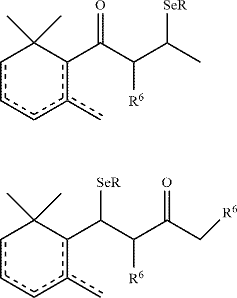 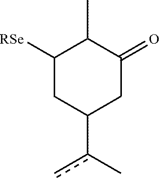 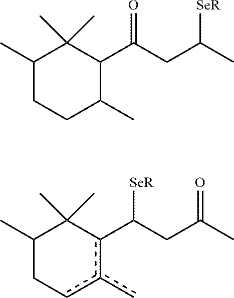 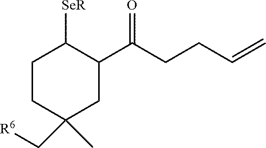 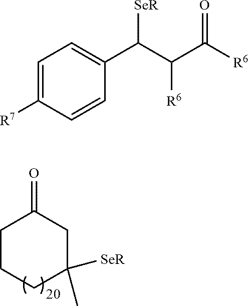 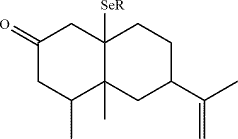 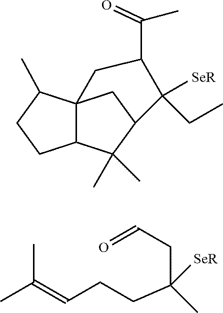 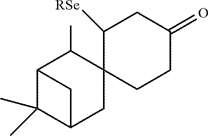 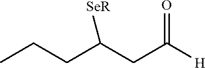
wherein the dotted line indicates the presence of one or two double carbon-carbon bonds between any adjacent pairs of the carbon atoms along which the dotted line extends;
R⁶ is a hydrogen atom or a methyl group; and
R⁷ is a hydrogen atom, a hydroxy group, a methoxy group or a linear or branched chain C₁-C₄ alkyl group.

9. A consumer product according to claim 8, wherein the selenide derivative has chemical formula: 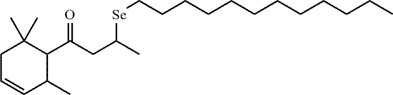 [SMILES: CCCCCCCCCCCC[Se]C(C)CC(=O)C1C(C)C=CCC1(C)C] 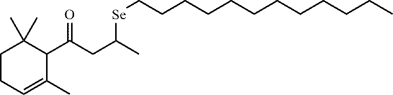 [SMILES: CCCCCCCCCCCC[Se]C(C)CC(=O)C1C(C)=CCCC1(C)C] 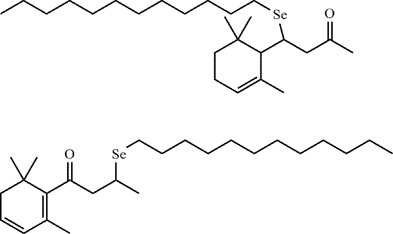 [SMILES: CCCCCCCCCCCC[Se]C(CC(C)=O)C1C(C)=CCCC1(C)C ; CCCCCCCCCCCC[Se]C(C)CC(=O)C1=C(C)C=CCC1(C)C] 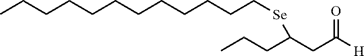 [SMILES: CCCCCCCCCCCC[Se]C(CC=O)CCC] 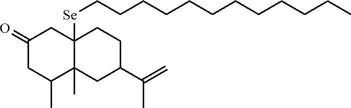 [SMILES: C=C(C)C1CCC2([Se]CCCCCCCCCCCC)CC(=O)CC(C)C2(C)C1] 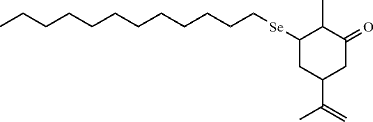 [SMILES: C=C(C)C1CC(=O)C(C)C([Se]CCCCCCCCCCCC)C1]
or 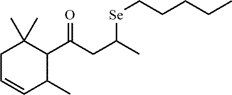 [SMILES: CCCCC[Se]C(C)CC(=O)C1C(C)C=CCC1(C)C]
.

10. A compound having the formula: 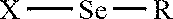
wherein X is a group generating a fragrance compound comprising an α,β-unsaturated aldehyde, an α,β-unsaturated ketone, an α,β-unsaturated ester, an α,β-unsaturated lactone or an α,β-unsaturated carboxylic acid by elimination of an ambient oxidation product of the group Se — R; and wherein R is a C₅₋₁₆ linear, cyclic or branched chain alkyl group, preferably wherein R is a C₁₀₋₁₄ linear alkyl group, more preferably a C₁₂ linear alkyl group.

11. A compound according to claim 10 of chemical formula: 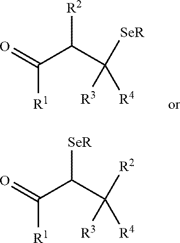
wherein R¹ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group or a C₁₋₁₂ alkoxy group; or
R¹ is a group — OR⁵, wherein R⁵ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group, or a C₁₋₁₂ alkoxy group; and
R², R³, R⁴ are independently a hydrogen atom; a C₁₋₂₀ linear or branched chain alkyl, alkenyl alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group optionally substituted with one or more of a substituent comprising a C₁₋₁₂ linear or branched chain alkyl group, a hydroxy or a C₁₋₁₂ alkoxy group; or
at least two of R¹, R², R³ and R⁴ are bonded together to form a saturated or unsaturated C₅₋₂₀ carbocyclic ring including the carbon atoms to which the at least two of R¹, R², R³, and R⁴ are attached, the ring being optionally substituted with one or more of a substituent comprising a C₁₋₁₂ linear, cyclic or branched chain alkyl or alkene group; or
when R¹ is the group —OR⁵, R⁵ and one of R², R³ and R⁴ are bonded together to form a saturated or unsaturated C₄₋₁₉ lactone ring including the carbon atoms to which the at least two of R², R³, R⁴and R⁵ are attached, the ring being optionally substituted with one or more of a substituent comprising a C₁₋₁₂ linear, cyclic or branched chain alkyl or alkene group.

12. A compound according to claim 11, wherein
R¹ is a hydrogen atom; a C₁₋₃ linear or branched chain alkyl group; or a C₅₋₁₀ cyclic alkene group, optionally substituted with one or more of a substituent comprising a C₁₋₃ linear or branched chain alkyl or alkene group; and
R², R³, R⁴ are independently a hydrogen atom; a C₁₋₅ linear or branched chain alkyl group; or a C₅₋₁₀ cyclic alkene group, optionally substituted with one or more of a substituent comprising a C₁₋₃ linear or branched chain alkyl or alkene group; or
at least two of R¹, R², R³ and R⁴ are bonded together to form a saturated C₅₋₂₀ carbocyclic ring including the carbon atoms to which the at least two of R¹, R², R³, and R⁴ are attached, the ring being optionally substituted with one or more of a substituent comprising a C₁₋₅linear or branched chain alkyl or alkene group.

13. A compound according to claim 11 , having chemical formula: 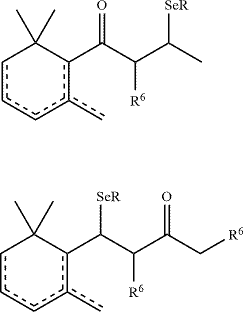 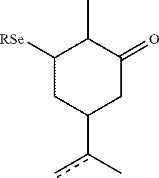 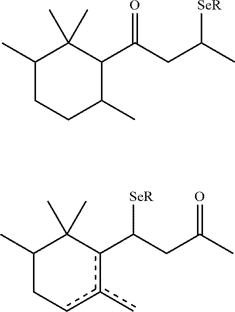 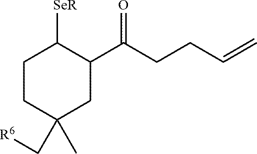 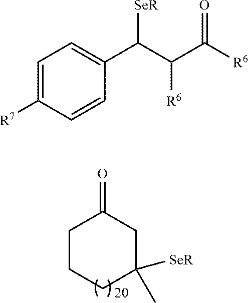 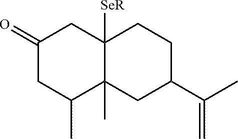 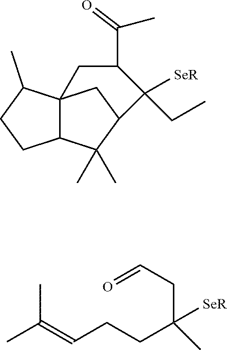 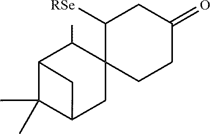 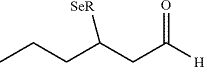
wherein the dotted line indicates the presence of one or two double carbon-carbon bonds between any adjacent pairs of the carbon atoms along which the dotted line extends;
R⁶ is a hydrogen atom or a methyl group; and R⁷ is a hydrogen atom, a hydroxy group, a methoxy group or a linear or branched chain C₁₋C₄ alkyl group.

14. A compound according to claim 13 having chemical formula 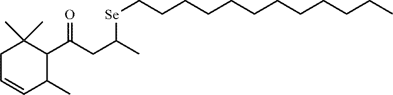 [SMILES: CCCCCCCCCCCC[Se]C(C)CC(=O)C1C(C)C=CCC1(C)C] 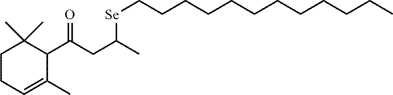 [SMILES: CCCCCCCCCCCC[Se]C(C)CC(=O)C1C(C)=CCCC1(C)C] 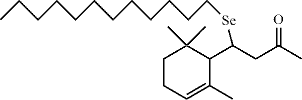 [SMILES: CCCCCCCCCCCC[Se]C(CC(C)=O)C1C(C)=CCCC1(C)C] 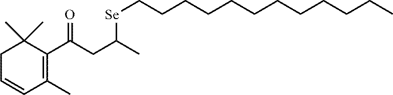 [SMILES: CCCCCCCCCCCC[Se]C(C)CC(=O)C1=C(C)C=CCC1(C)C] 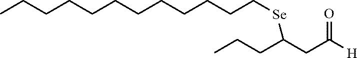 [SMILES: CCCCCCCCCCCC[Se]C(CC=O)CCC] 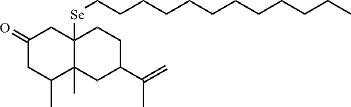 [SMILES: C=C(C)C1CCC2([Se]CCCCCCCCCCCC)CC(=O)CC(C)C2(C)C1] 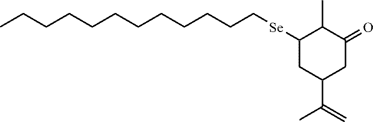 [SMILES: C=C(C)C1CC(=O)C(C)C([Se]CCCCCCCCCCCC)C1] 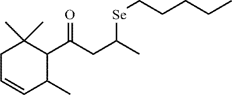 [SMILES: CCCCC[Se]C(C)CC(=O)C1C(C)C=CCC1(C)C]
.

15. A method for the preparation of a selenide derivative of a fragrance compound according to claim 10, comprising an α,β-unsaturated aldehyde moiety, an α,β-unsaturated ketone moiety, an α,β-unsaturated ester moiety, an α,β-unsaturated lactone moiety or an α,β-unsaturated carboxylic acid moiety having the formula 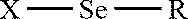
wherein X is a group generating a fragrance compound comprising an α,β-unsaturated aldehyde, an α,β-unsaturated ketone, an α,β-unsaturated ester, an α,β-unsaturated lactone or an α,β-unsaturated carboxylic acid by elimination of an ambient oxidation product of the group Se — R; and wherein R is a C₅₋₁₆ linear, cyclic or branched chain alkyl group, preferably wherein R is a C₁₀₋₁₄ linear alkyl group, more preferably a C₁₂ alkyl group;
the method comprising reacting the fragrance compound with an alkyl, aryl or heterocyclic selenide ion formed by treatment of a dialkyl, diaryl or diheterocyclic diselenide with borohydride; wherein the dialkyl, diaryl or diheterocyclic diselenide has chemical formula: 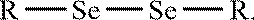
.

16. A method according to claim 15, wherein the α,β-unsaturated aldehyde, ketone, ester, lactone or carboxylic acid moiety has a chemical formula: 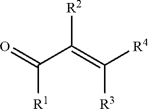
wherein R₁ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group or a C₁₋₁₂ alkoxy group; or
R¹ is a group — OR⁵, wherein R⁵ is a hydrogen atom; a C₁₋₂₀ linear or branched chain alkyl, alkenyl, alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group, optionally substituted with one or more of a substituent comprising a C₁₋₁₂ linear or branched chain alkyl group, a hydroxy group, or a C₁₋₁₂ alkoxy group; and
R², R³, R⁴ are independently a hydrogen atom; a C₁₋₂₀ linear or branched chain alkyl, alkenyl alkadienyl or alkynyl group; or a C₅₋₁₀ cyclic alkyl or cyclic alkene group, each optionally substituted with one or more of a substituent comprising a C₁₋₁₂ linear or branched chain alkyl or alkene group; or a C₆₋₁₀ aryl group optionally substituted with one or more of a substituent comprising a C₁₋₁₂ linear or branched chain alkyl group, a hydroxy or a C₁₋₁₂ alkoxy group; or
at least two of R¹, R², R³ and R⁴ are bonded together to form a saturated or unsaturated C₅₋₂₀ carbocyclic ring including the carbon atoms to which the at least two of R¹, R², R³, and R⁴ are attached, the ring being optionally substituted with one or more of a substituent comprising a C₁₋₁₂ linear, cyclic or branched chain alkyl or alkene group; or
when R¹ is the group —OR⁵, R5 and one of R², R³ and R⁴ are bonded together to form a saturated or unsaturated C₄₋₁₉ lactone ring including the carbon atoms to which the at least two of R², R³, R⁴ and R⁵ are attached, the ring being optionally substituted with one or more of a substituent comprising a C₁₋₁₂ linear, cyclic or branched chain alkyl or alkene group.
